(19) United States
(12) Patent Application Publication
(10) Pub. No.: US 2017/0203159 A1
(43) Pub. Date: Jul. 20, 2017

(54) Diving and swimming goggles

(71) Applicant: Klaus Schuwerk, Berlin (DE)

(72) Inventor: Klaus Schuwerk, Berlin (DE)

(21) Appl. No.: 15/002,358

(22) Filed: Jan. 20, 2016

(51) Int. Cl. A63B 33/00; A61F 9/02

(52) U.S. Cl. CPC A63B 33/002; A61F 9/022; A63B 2033/004; A61F 2009/021

(57) ABSTRACT

The invention relates to a pair of diving and swimming goggles with a device for automatic pressure equalization of the goggles' airspace. For this purpose, a pair of diving and swimming goggles is provided which includes an elastic membrane and an opening, wherein the elastic membrane forms a compressible air space and the opening is adapted to let water pass, which moves the elastic membrane and compresses the air space.

### FIELD OF USE

The invention relates to a pair of diving and swimming goggles with a device for automatic pressure equalization of the airspace inside the goggles. For this purpose, a pair of diving and swimming goggles according to claim 1 is provided.

### BACKGROUND OF THE INVENTION

When a swimmer or diver dives, the hydrostatic pressure increases inside the diving and swimming goggles and a negative pressure relative to the environment is generated. If this negative pressure is not equalized, redness and bleeding of the conjunctiva of the eyes occur (barotrauma of the eyes).

Swimming goggles are commonly worn to protect the eyes from chlorine and bacteria and for better underwater visibility. Their disadvantage is that they cannot be equalized. Lower barotraumas often occur even when swimming. To a greater extent this is true if they are improperly used for diving. Already at a depth of 3 to 5 metres barotraumas of the eyes usually occur.

Therefore, the British standard for swimming goggles (BS 5883: 1996) demands the following notice inside instruction manuals: “FOR SURFACE USE ONLY”.

When scubadiving or freediving, the equalization of the diving mask is either done via the nostril (conventional diving mask with integrated nose) or by selfequalizing diving goggles that have an additional compressible air volume (e.g. U.S. Pat. No. 2,182,104).

The disadvantage of a conventional diving mask is that a freediver uses valuable air from the lungs to equalize the mask. Instead, this air could be used to equalize the ears and to allow a longer duration of the dive.

Self equalizing diving goggles that have an additional compressible air volume have the disadvantage of a relatively large additional air volume that must be taken along in order to dive to greater depths.

The following example is mentioned in order to illustrate the problem: Freedivers, in the discipline Constant Weight (CWT), currently dive to depths around 120 m (i.e. an ambient pressure of about 13 bar). A low volume diving mask (air volume about 140 ml) and a pair of swimming goggles (air volume about 80 ml) would need, according to Boyle's Law (inverse relationship of pressure and volume of a gas), 140 ml ×13=1,820 ml and 80×13 ml=1040 ml of additional air to be carried along.

Therefore, existing solutions either lead to lower depths or impractical solutions, since the air volume is too large, unwieldy and also produces exceeding buoyancy. Even for a highly experienced freediver, using air from the lungs is only feasible to depths around 50 metres. Furthermore, the continuous equalization of the mask leads to additional stress for the freediver. For these reasons, competition freedivers dive either entirely without goggles or use so-called fluid-goggles (fluid-filled swimming goggles with integrated strong convex lenses), however these do not provide good visibility and have poor comfort, as the eyes are in contact with liquid. The second existing solution with an additional compressible air volume has not led to any practical solution because of buoyancy, poor hydrodynamics and instability while swimming.

### PURPOSE OF THE INVENTION

The purpose of the invention is to provide a pair of diving and swimming goggles, which require no pressure equalization, should be comfortable to wear and provide a good over-and underwater vision with a wide field of view. They should have good hydrodynamics and should remain dimensionally stable at swimming speeds of up to 4 metres per second. The invention should avoid the incident of barotraumas of the eyes, while using it inside the depth range. Last but not least, the diving and swimming goggles should not expose the user to any risk due to malfunction or improper use.

### DESCRIPTION OF THE INVENTION

The present diving and swimming goggles, according to claim 1, satisfy the requirements above.

A pair of diving and swimming goggles is provided, consisting in:

an elastic membrane; and an opening; wherein the elastic membrane is at least partially forming a compressible air space and that the opening is allowing water to pass. Consequently, the water moves the elastic membrane and compresses the air space.

Preferably, the opening is adjusted to let water pass when hydrostatic pressure increases.

It is obvious that the airspace can be partially formed by non-movable and/or non-stretchable material. It is also obvious that only a portion of the membrane can move and/or stretch under pressure, while the other portion remains unmoved and/or unstretched. Thus, the elastic membrane forms, at least partially, a compressible air space.

The term ‘moving the elastic membrane’ means a moving and stretching of the elastic membrane. This moving can be an exclusive moving of the elastic membrane, i.e. without stretching, including movements such as folding and eversion. It can also be the case of only an elastic stretching of the elastic membrane. Preferably both, moving and stretching of the elastic membrane occurs.

Diving and swimming goggles according to the present invention provide at least one air space in front of the user's eyes and may include a lens; a dimensionally stable shell; a contact area; an optional nose connection; an optional attachment system; an elastic membrane; and an opening.

The lens may consist, as in a conventional diving mask, of one piece or, as in swimming goggles, of separate pieces. The nostril can either be integrated into the air system of the goggles or not. Diving and swimming goggles according to the invention can either be used only for swimming, only for diving, or for both swimming and diving.

With increasing hydrostatic pressure, water can pass through an opening into the area of the membrane and thus compress the air space in front of the eyes.

The invention has accordingly-in contrast to previous solutions-no remaining rigid rest space which can contrast the ambient pressure. Therefore, when diving and swimming within its intended depth range, the invention does not generate dangerous negative pressure for the eye. At depth, only a small residual amount of air remains at the same pressure as the environment.

An advantage of the invention over a conventional diving mask is that the diver does not have to equalize the pressure inside the goggles and therefore can perform his dive in a relaxed manner. Another advantage, over the prior art, is that the air chamber can be protected by a dimensionally stable shell and allows therefore swimming speeds of up to 3.0 m/s and more. In addition, compared to the prior art, a lower initial and final volume can be obtained, and can therefore have a good hydrodynamic shape without substantial buoyancy.

In addition, with the aid of a nose clip the diver can equalize the ears without bringing the hand to the nose, which again greatly improves hydrodynamics, especially when using a monofin.

Another advantage is the easy handling and comfort while wearing them, as no liquids come into contact with the eye. Further advantages over existing solutions are the excellent underwater visibility and the wider field of vision due to the small distance between the lens and the eyes.

For pure swimming, the invention has the advantage that it prevents a barotrauma of the eyes, without having to renounce the good underwater visibility and the protection of the eyes from chlorine and bacteria.

Last but not least, the simplicity of its functioning guarantees a completely safe use within the intended depth range.

Furthermore, by adding a valve (for example, a small gap in the membrane), which at a specific negative pressure allows small amounts of water to pass, both security and range of the goggles can be increased.

Diving and swimming goggles, according to this invention, are a device intended for use in water, providing an air space in front of the user's eyes and usually consist of at least one lens, a contact area with the user's face and optionally of a dimensionally stable shell, a nose connection and a fastening system.

Swimming, according to this invention, is any swimming in which the head of the swimmer is submerged in water and therefore exposed to water pressure. Usually, a swimmer unintentionally dives while swimming. Typically, competitive swimmers at the starting jump and at the turn dive to depths around 2 metres.

Diving, according to this invention, includes diving to depths of more than 1 metre, as for example during freediving and scuba diving.

The elastic membrane or element of this invention may be made for example of a plastic material. Silicone rubber is especially suitable and preferred. The elastic membrane is adapted to move under hydrostatic pressure and/or to stretch or deform. Stretching, as used here, includes the property of the plastic material to extend reversibly, at least 1%, in a first and/or a second spatial direction. The first and the second spatial direction form in this case a surface of the elastic membrane. Preferably, the elastic membrane can be stretched reversibly by at least 5%, in a first and/or a second spatial direction, more preferably by at least 10% in a first and/or a second spatial direction,

by at least 30% in a first and/or a second spatial direction

by at least 50% in a first and/or a second spatial direction,

by at least 80% in a first and/or a second spatial direction,

by at least 100% in a first and/or a second spatial direction,

by at least 200% in a first and/or a second spatial direction,

or by at least 300% in a first and/or a second spatial direction.

Dimensionally stable, according to this invention, includes the property of the original shape to remain essentially unchanged, even with higher speeds under water, preferably at speeds of ≧0.5 m/s, ≧1 m/s, ≧2 m/s or ≧3 m/s. Essentially unchanged, according to this invention, preferably includes a deformation of less than 10 mm, more preferably ≦5 mm, ≦4 mm, ≦3 mm, ≦2 mm ≦1 mm, most preferably less than 0.5 mm. Deformation, according to this invention, includes the moving of an object's point from its original position in any direction due to an external force.

### DESCRIPTION OF THE DRAWINGS

The invention's illustrative, non-limitative drawings show:

FIG. 1 is a perspective view of an embodiment of the invention;

FIG. 2 is a view of an embodiment of the invention;

FIG. 3 is a sectioned view of an embodiment of the invention at an uncompressed state;

FIG. 4 is a horizontal section through an embodiment of the invention at an uncompressed/compressed state;

FIG. 5 shows a vertical section through an embodiment of the invention at an uncompressed/compressed state;

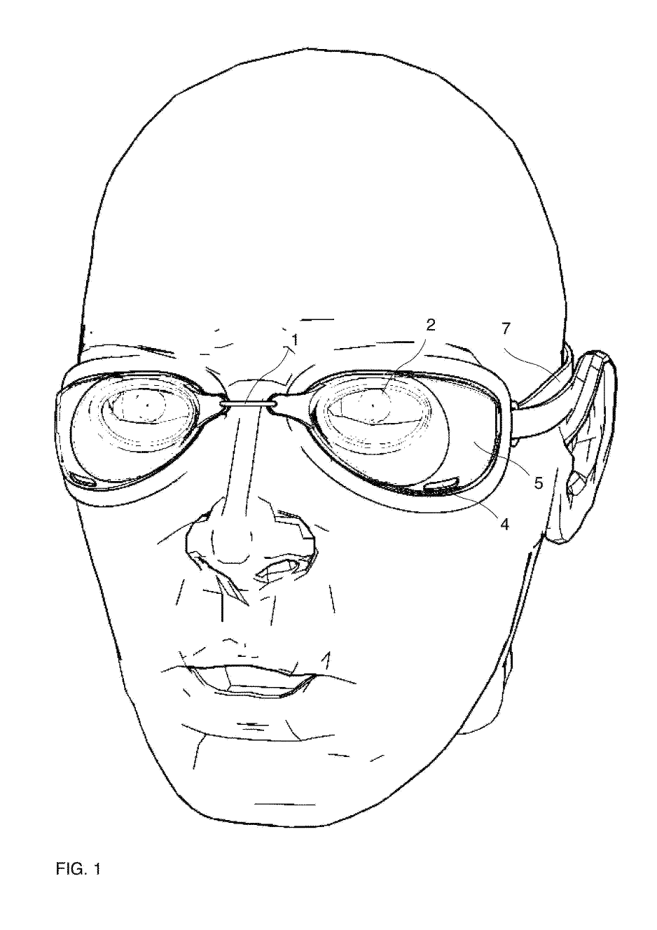

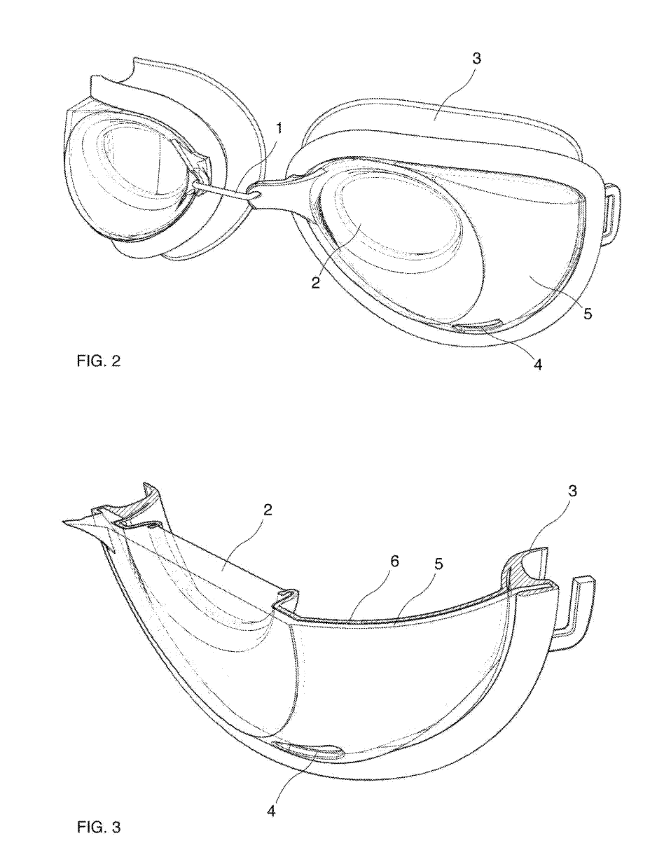

### DETAILED DESCRIPTION OF THE INVENTION

According to one preferred embodiment, a pair of diving and swimming goggles is provided. The diving and swimming goggles can include a lens; a dimensionally stable outer shell; a contact area; a nose connection; a fastening system; an elastic membrane and an opening, characterized in that the elastic membrane is forming a compressible air space and the opening is adapted to let water pass with increasing hydrostatic pressure, which moves the elastic membrane and compresses the air space.

Preferably, the membrane is made of an elastic material which has an elongation at break of more than 30%, more preferably ≧50%, ≧100%, ≧150%, ≧200, ≧300%, ≧400%, ≧500% , ≧600%, ≧700%, ≧800%, most preferably ≧900%. The elongation at break can be determined, for example, in accordance to DIN 53504-S1. The thickness and geometry of the membrane and the elasticity of the material is preferably chosen in such a way that, within the depth range, it will preferably not result in any negative pressure of more than 50 mbar, more preferably not more than 40 mbar, not more than 30 mbar, not more than 20 mbar, most preferably not more than 10 mbar. A person skilled in the art is able to make appropriate calculations and estimations for the case.

To a person skilled in the art, it is further clear that the opening is preferably large enough to allow water to pass at a sufficient speed, so that the above mentioned negative pressure is not exceeded.

To a person skilled in the art, it is further clear that the opening can be shaped in a different manner (for example, many small holes, water-permeable membrane, etc.). Preferably when choosing the opening's design, the stability of the goggles, while swimming at speeds of up to 3 m/s or more, should be taken into account.

The lens and the dimensionally stable shell of the invention can be made of any suitable material in one or more parts. Preferably in transparent plastic material.

Appropriate plastic materials are well known to the person skilled in the art.

One or two lenses can be used. Two lenses are preferred due to the resulting lower internal volume. The lens should be as close as possible to the user's eye. The distance between eye and lens is preferably ≦30 mm, more preferably ≦20 mm, ≦10 mm, ≦9 mm, ≦8 mm, ≦7 mm, ≦6 mm, ≦5 mm, ≦4 mm, ≦3 mm, ≦2 mm, most preferably ≦1 mm. Interchangeable lenses of different thicknesses can adapt the goggles to different faces and eye shapes.

According to this invention, the contact area is the contact surface of the diving and swimming goggles on the user's face. It lies sealingly on the skin and prevents the penetration of water into the air space. The contact area is preferably made of elastic plastic. Suitable plastic materials are well known to the person skilled in the art. Alternatively, a non-flexible contact area, as in the so-called ‘swedish goggles’, is also possible.

Optionally, an adjustable nose connection allows adaptation to the individual eye distance of the user. Alternatively, other common systems such as interchangeable nose bridges can be used. The person skilled in the art is aware of such systems.

According to a further embodiment, a dimensionally stable shell covers the elastic membrane completely or partially.

According to yet another embodiment, the opening is arranged in the dimensionally stable shell.

According to a further embodiment, the diving and swimming goggles comprise at least a second compressible air space, which is located outside the dimensionally stable shell and is connected to the air space around the eyes.

According to a further embodiment, the diving and swimming goggles comprises a lens.

According to a further embodiment, the lens is not flat, but adapted to the geometry of the eye area in order to leave a minimum residual air space around the eyes. Consequently, the inner side of the lens is in principle a negative mould of the eye cavity. The outer surface of the lens can either be flat with a variable cross-section or follow with constant thickness the shape of the orbit. The lens can also be custom-made for an individual face.

According to another embodiment, the lens is equipped with an eye correction factor for eyeglass wearers. With this, all usual refractive errors can be corrected, as with glasses outside water. The lens may be, for example, bi-convex, plano-convex, concave-convex, bi-concave, plano-concave or convex-concave in various strengths.

According to a further embodiment, the diving and swimming goggles have two lenses, which are plane and parallel to each other in order to allow a distortion free vision under water, which is especially important for spearfishing.

According to yet another embodiment, the lens has a protective anti-fog-coating and/or a scratch-protection and/or UV-protection and/or mirror-effect and/or color tone. The anti-fog coating prevents air from condensing on the inside of the lens. The scratch protection prevents scratching of the lens both on the inside and on the water side. Suitable materials are known to the person skilled in the art. Other known types of coatings and materials modification are possible.

According to yet another embodiment of the invention, a device is included which lets small amounts of water into the goggles from a threshold of about 30 mbar vacuum until the vacuum is again below this threshold. The threshold is preferable ≦29 mbar, more preferably ≦28 mbar, ≦27 mbar, ≦26 mbar, ≦25 mbar, ≦24 mbar, ≦23 mbar, ≦22 mbar or ≦21 mbar. Most preferred is a threshold of ≦20 mBar.

A slot in the wall of the air space is formed in such a way that it opens automatically by the higher external pressure. Other known valve solutions are also possible. The person skilled in the art is aware of such valve solutions. The threshold value is chosen in such a way that no barotrauma can occur.

Further preferred embodiments of the invention are specifically intended for swimming. The elastic membrane can form here the air space only partially, so that inside the intended depth range no negative pressure occurs. Preferred portion of the air space's surface (excluding the face), formed by the membrane, is between 5%-80%. Preferred depth ranges of specific embodiments, intended for swimming, are 0-1 m, 0-2 m, 0-3 m, 0-4 m and 0-5 m.

Other preferred embodiments of the invention are intended for swimming or diving to different depth ranges. Preferred depth ranges are 0 to 1 m, 0 to 2 m, 0 to 3 m, 0 to 4 m, 0 to 5 m, 0 to 7 m, 0 to 10 m, 0 to 20 m, 0 to 30 m, 0 to 40 m, 0 to 50 m, 0 to 75 m, 0 to 100 m and 0 to more than 100 m.

According to another embodiment, the goggles according to this invention are intended to be used as diving and swimming goggles.

Another preferred embodiment of the invention, as a non-limiting example, is described below in detail.

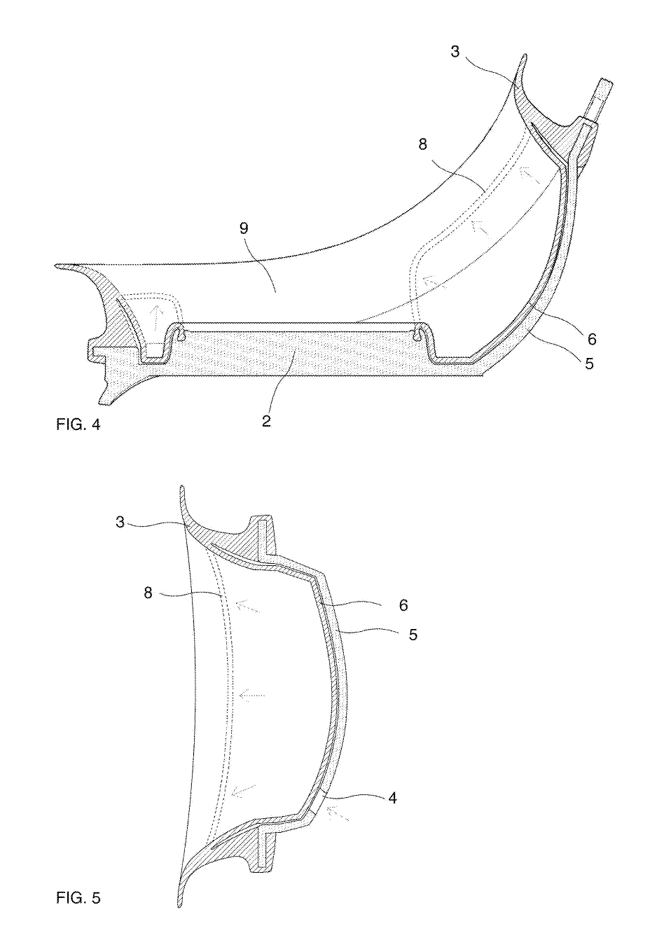

The preferred embodiment of the invention consists of a dimensionally stable outer shell 5 with an integrated transparent lens 2 and an opening 4, preferably manufactured in polymethyl methacrylate (PMMA). A compressible air space 9 is formed by an elastic membrane 6 and a soft contact area 3, preferably made in platinum-catalyzing liquid silicone rubber. Next, the preferred embodiment includes an adjustable nose connection 1 and an elastic fastening system around the head 7. FIG. 4 shows a horizontal section through the eye area. FIG. 5 shows a vertical section through the area of the dimensionally stable outer shell. The lens 2, the elastic membrane 6 and the contact area 3 form the compressible air space 9. When diving, water passes through one or more openings 4 into the space between the dimensionally stable shell 5 and the elastic membrane 6. The membrane 6 can move in this way to position 8, which prevents from dangerous negative pressure in the air space 9.

Below is described one possible way to manufacture the goggles according to the invention. To the person skilled in the art, it is obvious that the goggles of the invention can also be realized through other manufacturing methods.

The lens 2 and the dimensionally stable shell 5 can be manufactured in one piece by injection molding of polymethyl methacrylate (PMMA). A simple mold of an upper and a lower part is suitable for this purpose.

The contact area 3 and the elastic membrane 6 can be made in one piece by injection molding of platinum-catalyzing liquid silicone rubber (LSR). The material has in this case preferably an elongation at break factor of at least 300% and a hardness between 10-80 Shore (A), preferably 40 Shore (A). The connection between the lens and the membrane has to be watertight, for example with a clip connection.

A normal monofilament in nylon can be used for the adjustable nose connection 1. Alternatively adjustable and non-adjustable nose connections can be used, as often seen in common swimming goggles.

The fastening system can be a simple rectangular profile (1.5 mm×8 mm) made of platinum-catalyzing liquid silicone rubber (LSR). The user can knot this to the correct length.

1. diving and swimming goggles, comprising:
an elastic membrane (6);
and an opening (4);
characterized in that the elastic membrane (6) is forming at least partially a compressible air space (9) and that the opening (4) is adapted to let water pass, which moves the elastic membrane (6) and compresses the air space (9).

2. diving and swimming goggles according to claim 1, comprising a dimensionally stable shell (5), which covers completely or partially the elastic membrane (6).

3. diving and swimming goggles according to claim 2, wherein the opening (4) is arranged in the dimensionally stable shell (5).

4. diving and swimming goggles according to any one of the preceding claims, wherein at least a second compressible air space is located outside of the dimensionally stable shell (5) and is connected to the air space (9).

5. diving and swimming goggles according to any one of the preceding claims, comprising a lens.

6. diving and swimming goggles according to claim 5, wherein the lens is adjusted to the geometry of the eye cavity.

7. diving and swimming goggles according to one of the claims 5 and 6 wherein the lens is provided with an eye correction factor for eyeglass wearers.

8. diving and swimming goggles according to one of the claims 5-7, wherein the lens is provided with an anti-fog protection and/or scratch-protection and/or UV protection and/or any other known coating or material modification.

9. diving and swimming goggles according to any one of the preceding claims, comprising a pressure equalizing valve.

10. Use of the diving and swimming goggles according to any of the preceding claims.
